(19) United States
(12) Patent Application Publication
(10) Pub. No.: US 2018/0355380 A1
(43) Pub. Date: Dec. 13, 2018

(54) METHODS AND KITS FOR QUALITY CONTROL

(71) Applicant: GENETICS RESEARCH, LLC, D/B/A ZS GENETICS, INC., Wakefield, MA (US)

(72) Inventors: Anthony P. Shuber, Wakefield, MA (US); William Glover, Wakefield, MA (US)

(21) Appl. No.: 15/877,614

(22) Filed: Jan. 23, 2018

Related U.S. Application Data
(60) Provisional application No. 62/568,136, filed on Oct. 4, 2017.
(60) Provisional application No. 62/526,091, filed on Jun. 28, 2017.
(60) Provisional application No. 62/519,051, filed on Jun. 13, 2017.

(51) Int. Cl. C12N 15/90; C12N 15/11; C12N 9/22; C12N 9/96; C12Q 1/6869

(52) U.S. Cl. CPC C12N 15/902; C12N 15/11; C12N 9/22; C12N 2800/80; C12Q 1/6869; C12N 2310/20; C12N 9/96

(57) ABSTRACT

Methods and kits are provided that are useful as quality controls for gene editing tools. When a gene editing process is proposed for some subject nucleic acid, the gene editing process may be performed on a representative sample—a sample that represents the subject nucleic acid. Off-target effects may be measured and shown for the representative sample to show a prospective rate of off-target activity were the gene editing process to be performed on the subject nucleic acid.

### CROSS-REFERENCE TO RELATED APPLICATIONS

This application claims the benefit of, and priority to, U.S. Provisional Application No. 62/568,136, filed Oct. 4, 2017, U.S. Provisional Application No. 62/526,091, filed Jun. 28, 2017, and U.S. Provisional Application No. 62/519,051, filed Jun. 13, 2017, the contents of each of which are incorporated by reference.

### TECHNICAL FIELD

The disclosure relates to methods and kits for quality control useful with nucleic acid enrichment or genome editing.

### BACKGROUND

Clustered regularly interspaced short palindromic repeats (CRISPR) and CRISPR-associated (Cas) genes are used by bacteria to eliminate genetic material of invading viruses. CRISPR/Cas systems have been developed for functions such as gene editing. Such gene editing systems have the potential to provide permanent, inheritable modification of genes within organisms. As such, CRISPR/Cas systems have many potential applications in medicine and agriculture. The technology had been used to inactivate genes in human cells, to study Candida albicans, to modify biofuel-producing yeasts, and to genetically modify crop strains.

Despite its promise, off-target effects are always a concern when using CRISPR/Cas. If a Cas nuclease and guide RNA are used to edit a locus of interest, there is the possibility that the nuclease may also make changes at off-target loci, with detrimental consequences for cell survival or development.

Current approaches to determining off-target rates for gene editing applications require that whole genome sequencing (WGS) be performed on the targeted genome of interest before and after gene editing, in order to determine the extent of off-target events. Although that approach may theoretically generate an absolute off-target rate for a Cas9/gRNA complex, within the sequencing field that there are a number of variables (e.g., sample prep, library production, depth of coverage, bioinformatic analysis) that can significantly differ from one clinic or group of investigators to another.

### SUMMARY

Methods and kits are provided for use as quality controls for gene editing. To establish that a gene editing process is suitable for a target nucleic acid such as a whole genome, the gene editing is performed on just a representative portion of the target. An off-target rate is measured for that portion and the measured off-target rate is used to predict off-target activity over the whole genome. Thus, methods of the invention include testing a gene editing system on just a portion of the target to show that the gene editing system will exhibit an acceptable level of off target activity when used in vivo.

The tested portion of the target nucleic acid functions as a representative sample of the entire target nucleic acid. The representative portion is a sample for which off-target gene editing activity is indicative of off-target activity for the target nucleic acid, e.g., in vivo on a human genome. The representative portion may include one or more nucleic acids that statistically represent the entire target, and may be provided as part of a kit of the invention, or may be obtained by enrichment or isolation from the target nucleic acid. The inventive quality control methods show off-target gene editing activity for the representative portion of a subject nucleic acid before the gene-editing is performed on the subject nucleic acid.

One benefit of testing the gene editing system on just a representative portion of the entire target nucleic acid is that it may be feasible to show an off-target rate for the smaller, less-complex representative portion than for the whole genome. For example, if off-target activity will be determined by sequencing the product of a gene editing process, using just a representative portion of the genome can avoid problems with whole genome sequencing such as difficulty in achieving a required level of coverage (e.g., 30x) across every locus being sequenced. Thus by testing gene editing on just a representative portion of the target, the rate of off-target activity can be detected and demonstrated rapidly and with greater accuracy than with an entire genome.

Additionally, measuring the off-target rate can help select parameters of a gene editing treatment so that, when performed in vivo, the gene editing treatment does not exhibit an unacceptable off-target rate. The quality control process may be performed in a series of trial runs with varying conditions to determine the conditions under which an acceptable off-target rate obtains. Thus, methods and kits of the invention may be used to determine a degree of off-target events that occur with a gene-editing procedure.

While kits and methods of some embodiments make use of a representative sample to show an acceptable off-target rate, those kits and methods may also be used to show that a proposed representative sample is useful as a representative sample, and that a proposed gene editing process will exhibit only an acceptable off-target rate when performed on the representative sample.

In some embodiments, sequence-specific polynucleotide capture and enrichment is used to obtain the representative portion of a target nucleic acid. A representative sample of the target nucleic acid is obtained. The representative portion is protected (e.g., in a sequence-specific fashion by, for example, a binding protein or complex such as a Cas endonuclease, TALE, etc.) while a balance of the target is digested, ablated, or removed.

In some applications, sequence-specific polynucleotide capture and enrichment is useful if it is shown that the sequence-specific capture is substantially free of off-target effects. For example, CRISPR/Cas may be used to capture and enrich target loci, which may be used in the context of sample preparation, when preparing a sample for sequencing or other analysis. In such applications, the target is enriched because it is of interest in research or medicine. For example, the target may include cancer biomarkers or genes implicated in hereditary disorders. When isolating or enriching some representative genetic material of interest, kits and methods of the invention are used to detect and show an off-target rate of the proposed gene editing system and thus show that the gene editing system is successful at obtaining a representative sample from a more complex, mixed biological sample.

In certain embodiments, the invention provides a universal CRISPR experimental QC kit and corresponding methods of quality control. Methods of the invention may be used to evaluate gene driven natural selection processes. Methods of the invention use a representative portion of a target to evaluate off-target activity. Demonstrating that the off-target rate for the representative portion is representative for the target may include calculating a “relevant off-target rate” or a “impact-adjusted off-target rate”. Methods and kits of the invention may be tailored to the application in recognition of the potential that what is representative may vary among organisms (e.g., human, plant, pathogen etc.) Such kits and methods may accordingly be standardized for a species of interest. Because the kits and methods demonstrate that a particular application of gene editing exhibits an acceptable off-target rate and results in a representative sample, the sample that results from the use of such kits and methods will also reduce the complexity of the experimental genome of interest. By obtaining a representative sample and reducing complexity of the genome of interest, prospective gene-editing processes may be readily shown to be adaptable for general, industrial, or clinical use.

A reduction in complexity will have a significant impact of the variables downstream in a whole-genome sequencing (WGS) process (e.g. informatics etc.) and will reduce inter lab variability. Performing a quality control that includes measuring off-target rate is useful for ensuring that the reduction of complexity is replicable. Additionally, the use of kits and methods to demonstrate an acceptable off-target rate satisfies regulatory needs and requirements within the diagnostic and therapeutic areas of investigation and clinical testing.

In certain aspects, the invention provides a method of quality control for a gene editing system. The method includes treating a representative portion of a target nucleic acid with a gene editing system to yield a nucleic acid product and sequencing the nucleic acid product to produce sequence reads. An off-target rate for the representative portion is determined by comparing the sequence reads to predicted sequence that would result from treatment by the gene editing system with no off-target activity. An off-target rate for the target nucleic acid is inferred based on the determined off-target rate for the representative portion. In some embodiments, the representative portion is less than the target nucleic acid. For example, the target nucleic acid may include a target of the gene editing system, which target is not within the representative portion. The gene editing system may include programmable nuclease such as a Cas endonuclease (e.g., Cas9, Cpf1, CasX, CasY) or a nucleic acid encoding the programmable nuclease. The representative portion may be obtained from the target nucleic acid by a process that is agnostic of genes or coding regions within the target nucleic acid.

The method may include sequencing the representative portion prior to the treating step to produce the sequence corresponding to no off-target activity, i.e., the “predicted sequence”. Optionally, the representative portion comprises one or more pre-determined segments of target nucleic acid provided in a kit.

In certain embodiments, the method includes performing an enrichment prior to the treating step to obtain the representative portion from the target nucleic acid. The enrichment may include protecting the representative portion in sequence-specific manner and digesting unprotected nucleic acid. The method may include obtaining the representative portion from the target nucleic acid by ablation of portions of the target nucleic acid that are not comprised by the representative portion. This may include protecting the representative portion (e.g., with sequence-specific binding proteins) while digesting other nucleic acid (e.g., with exonuclease).

The method may include treating the representative portion with the gene editing system in several trials under unique conditions, showing an inferred off-target rate for each trial. and selecting a set of conditions that gives an off-target rate below a threshold. The threshold may be a predetermined threshold indicating that the gene editing system used under the selected conditions will not exhibit a significant off-target rate when performed on the target nucleic acid.

In certain embodiments, the method is used as a quality control for a use of Cas9, and the method includes introducing Cas9 and a guide RNA to the representative portion, determining the off-target rate for Cas9 with the representative portion, and reporting an inferred off-target for Cas9 with the target nucleic acid based on the determined off-target rate for binding of Cas9 and the guide RNA to the representative portion.

Aspects of the invention provide a method that includes treating a representative portion of a target nucleic acid with a gene editing system to yield a nucleic acid product, determining an off-target rate for the representative portion by performing an assay on the nucleic acid product, and inferring an off-target rate for the target nucleic acid based on the determined off-target rate for the representative portion. Preferably, the representative portion is less than the target nucleic acid. The gene editing system may include a Cas endonuclease and a guide RNA. The method may include obtaining the representative portion from the target nucleic acid by ablation of portions of the target nucleic acid that are not comprised by the representative portion. The method is useful as a quality control process for Cas9 endonuclease to show an acceptable rate of off-target activity by the Cas9 endonuclease with the target nucleic acid. Optionally, the target nucleic acid includes a human genome. Determining the off-target rate may include sequencing a product of the process, a heteroduplex cleavage assay, a high-resolution melt curve, qPCR; or a heteroduplex mobility assay.

In some aspects, the invention provides a quality control method. The method includes providing material designed to interact with a target in a subject nucleic acid, introducing the material to a representative sample for the subject nucleic acid, and determining an off-target rate of interaction of the material with the representative sample. An off-target rate of interaction between the material and the subject nucleic acid may be projected based on the determined off-target rate of interaction of the material with the representative sample. Determining the off-target rate may include performing an assay to show an amount of off-target activity resulting from introduction of the material to the representative sample. Such an assay may include, for example, sequencing a product of the process; a heteroduplex cleavage assay (using, e.g., T7 endonuclease I); a high-resolution melt curve; qPCR; or a heteroduplex mobility assay.

In some embodiments, the representative sample include one or more pre-determined fragments of test nucleic acid provided in a kit. Alternatively, providing the representative sample may include enriching the representative sample from the subject nucleic acid.

In certain genome-editing embodiments, the material is designed to interact with the target to perform gene editing on the subject nucleic acid. Preferably, the materials include at least one programmable nuclease—or a nucleic acid encoding the programmable nuclease—such as a Cas endonuclease. Such embodiments may include reporting an off-target rate of binding of the programmable nuclease or Cas endonuclease to the subject nucleic acid based on the determined off-target rate for the representative sample.

In certain enrichment embodiments, the material is designed to interact with the target to perform enrichment of a target nucleic acid from the subject nucleic acid. The enrichment process may include protecting the target nucleic acid in sequence-specific manner and cleaving unprotected nucleic acid. Determining the off-target rate for the process shows that only the target nucleic acid survives the cleaving step.

Methods may include conducting at least a first trial, second trial, and third trial of introducing the material to the representative sample, each trial comprising unique conditions; showing a respective off-target rate for each trial; and selecting a set of conditions associated with an off-target rate shown to be below a threshold. The threshold may be a predetermined threshold indicating that the process performed under the selected conditions will not exhibit a significant off-target rate when performed on the subject nucleic acid.

Embodiments of the invention use the method as a quality control for a use of Cas endonuclease. Such embodiments of the method include introducing a Cas endonuclease and at least one guide RNA to the representative sample; determining the off-target rate for binding of the Cas endonuclease and the guide RNA to the representative sample; and reporting an off-target rate for binding of the Cas endonuclease and the guide RNA to the subject nucleic acid based on the determined off-target rate for binding of the Cas endonuclease and the guide RNA to the representative sample.

Certain embodiments of the invention provide a quality control (QC) method for use with an in vivo gene editing procedure. With in vivo gene editing, an enrichment is performed to obtain a representative portion of a target nucleic acid from a subject, such as an organism for whom gene editing will be performed. The representative sample of the subject's nucleic acid may be obtained using a negative enrichment technique in which the enrichment comprises taking a sample from the subject and protecting the representative portion in sequence-specific manner and digesting unprotected nucleic acid. The representative portion may be protected using a gene-editing system. Preferably, the gene editing system comprises programmable nucleases (or nucleic acid encoding the programmable nuclease), and in a most preferred embodiment, each programmable nuclease includes Cas endonuclease (e.g., complexed to a guide RNA specific to part of the representative portion. Exonuclease is introduced into the sample to digest everything but the protected representative portion. The remaining DNA (e.g., the representative portion) is extracted for sequence (e.g., via standard nucleic acid extraction techniques). The in vivo QC method includes sequencing the representative portion to obtain pre-Tx sequence reads (aka expected sequence). The gene editing procedure is performed on the subject. After the gene editing is performed on the subject, another enrichment is performed to obtain a post-treatment representative portion. The in vivo QC technique includes sequencing the post-treatment representative portion to obtain post-Tx sequence reads. The post-Tx sequence reads are compared to the pre-Tx sequence reads to determine an off-target rate. In an exemplary embodiment, any significant difference between the pre-Tx sequence reads and the post-Tx sequence reads counts as evidence of an instance of off-target activity. The evinced instances of off-target activity may be counted to determine an off-target rate for the representative portion. The in vivo QC method may preferably include inferring an off-target rate for the target nucleic acid based on the determined off-target rate for the representative portion.

Aspects of the invention provide kits for quality control. An exemplary kit may include a representative sample of a subject nucleic acid, reagents for obtaining the representative sample, instructions for determining an off-target rate of a gene editing process on the representative sample, or combinations thereof.

Aspects of the invention provide methods and kits for genomic capture, e.g., for targeted genomic capture or the capture and isolation of specific targeted genomic regions—which capture methods may be used in quality control methods. In certain aspects, the invention provides a method that includes selectively protecting a target nucleic acid and degrading unprotected, non-target nucleic acids, thereby facilitating analysis of the target nucleic acid. Selectively protecting the target nucleic acid may include introducing into a sample at least one molecule that selectively protects the target nucleic acid from digestion in a sequence-specific manner. Degrading the non-target nucleic acids may include digesting the non-target nucleic acids with an exonuclease. The method may further include capturing the target nucleic acid on a substrate for the analysis. In some embodiments, the target nucleic acid is at least a few kilobases in length. The degrading step may degrade a plurality of non-target nucleic acid fragments.

Optionally, the method includes analyzing the target nucleic acid to determine a sequence (e.g., by next-generation sequencing). The sequence may be determined by single-molecule sequencing. In certain embodiments, the target nucleic acid is not amplified and the determined sequence is at least a few kilobases in length. Preferably, the analysis leaves the target nucleic acid intact for a subsequent analysis.

In some embodiments, the at least one molecule includes: a nuclease that cleaves the target nucleic acid and leaves an end with an overhang; a polymerase that fills in the end; and one or more modified nucleotides that are added to the end by the polymerase, wherein the modified nucleotides resist digestion. In preferred embodiments, the at least one molecule includes: a complex comprising an RNA-guided nuclease and a guide RNA (e.g., Cas9 & gRNA), wherein the complex binds to the target nucleic acid and inhibits a second nuclease from digesting the target nucleic acid. In certain embodiments, the at least one molecule comprises a DNA-binding protein.

In a related aspect, the invention provides a kit that includes at least one molecule that selectively protects a target nucleic acid from digestion; and a nuclease that digests un-protected, non-target nucleic acid. The at least one molecule may include, for example, an RNA-guided nuclease, a primer, a polymerase, a modified nucleotide, and/or a DNA binding protein.

### BRIEF DESCRIPTION OF THE DRAWINGS

FIG. 1 diagrams a QC method.

FIG. 2 shows a gene editing system.

FIG. 3 shows enrichment to obtain a representative portion of a target nucleic acid.

FIG. 4 illustrates a kit for quality control according to one embodiment.

FIG. 5 diagrams a quality control method.

FIG. 6 illustrates assaying for on- and off-target interactions.

FIG. 7 shows the result of fragmenting or amplifying products of the assay.

FIG. 8 illustrates determining an off-target rate.

FIG. 9 shows negative enrichment using a programmable nuclease.

### DETAILED DESCRIPTION

In an illustrative embodiment, methods of the invention use a kit and the kit includes the representative sample. The kit may be provided as a quality control kit for gene editing projects in a given species, such as human. Where a gene editing process may be proposed for use in human subject, the relevant subject nucleic acid may be a human genome (and optionally a mitochondrial genome). For such applications, it may be useful to provide a kit that includes, as a representative sample, a set of human genetic material that is less than, but stands for, a human genome. For example, the representative sample may include fragments of DNA that themselves include portions of a combination of introns, exons, regulatory regions, telomeres, junk DNA, and other features. The proposed gene editing tools (e.g., Cas endonuclease and guide RNA(s)) may be introduced to the representative sample from the kit. A portion of the resulting product may be run out on a gel (e.g., by polyacrylamide gel electrophoresis (PAGE)). A second lane of the gel can have a portion of the representative sample that was not exposed to the gene editing reagents. Each lane will produce a ladder. Comparison of the ladders gives an estimate of off target activity by the Cas endonuclease. In preferred embodiments, the representative sample include features from a human genome (or other species) but specifically exclude the intended target of the gene editing reagents.

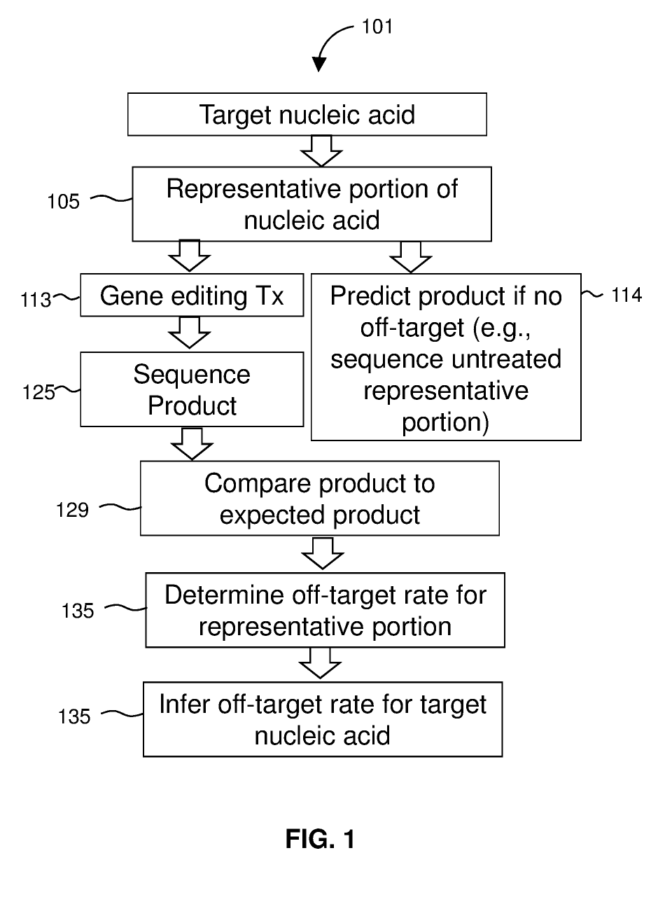

FIG. 1 diagrams a QC method 101. The method 101 includes obtaining 105 a representative portion of a target nucleic acid. The obtaining 105 may be done by a negative enrichment technique, for example, or the representative portion may be provided as part of a standardized kit. The method 101 includes treating 113 the representative portion of the target nucleic acid with a gene editing system to give a nucleic acid product. The product of the treatment 113 is sequenced 125 to produce sequence reads. An expected sequence is provided 114 (either predicted or by sequencing an aliquot of the representative portion that is not treated with the gene editing system). The sequence reads are compared 129 to the expected product to determine 135 an off-target rate for the representative portion. The method 101 includes inferring 139 an off-target rate for the target nucleic acid based on the determined off-target rate for the representative portion.

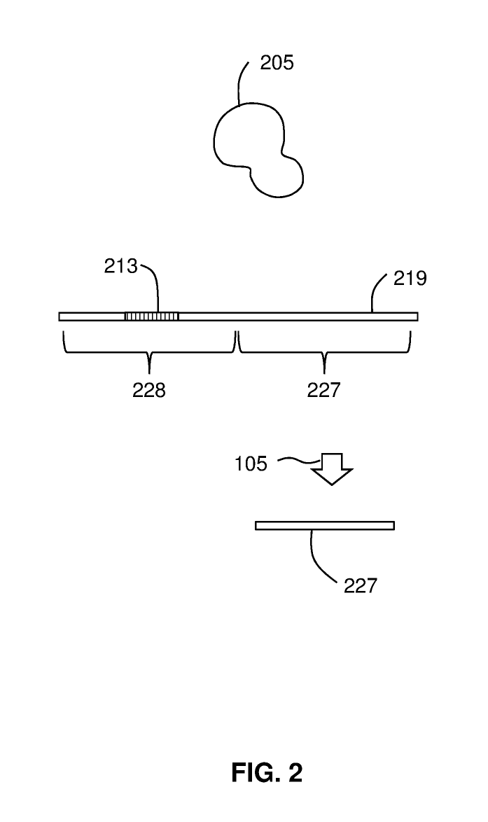

FIG. 2 shows a gene editing system 205 designed to interact with a target 213 in a target nucleic acid 219. In preferred embodiments, the representative portion 227 of the target nucleic acid 219 is a fragment of, or subset of, the target nucleic acid 219. In certain preferred embodiments, the gene editing system 205 includes a programmable nuclease (e.g., TALENs, ZFN, meganuclease) and most preferably includes a Cas endonuclease, e.g., Cas9.

Embodiments of the invention include obtaining 105 the representative portion 227 from the 219. Any suitable technique may be used to obtain 105 the representative portion such as, for example, amplification (e.g., PCR), fragmentation, hybrid capture (e.g., with probes), etc. In preferred embodiments, the representative portion 227 is obtained 105 from the target nucleic acid by a negative enrichment technique in which segments 228 of the target nucleic acid 219 other than the representative portion 227 are ablated away (e.g., promiscuously digested by a nuclease).

For enrichment of the representative portion 227, a sample may be obtained that includes the target nucleic acid 219. Typically, the target nucleic acid 219 includes the target 213 as well as any number of off-target regions. In a preferred embodiment, the gene editing system 205 is designed to interact with the target to perform gene editing on the subject nucleic acid. Using techniques described below,

Obtaining 115 the representative portion 227 may include enriching the representative portion 227 from the target nucleic acid 219.

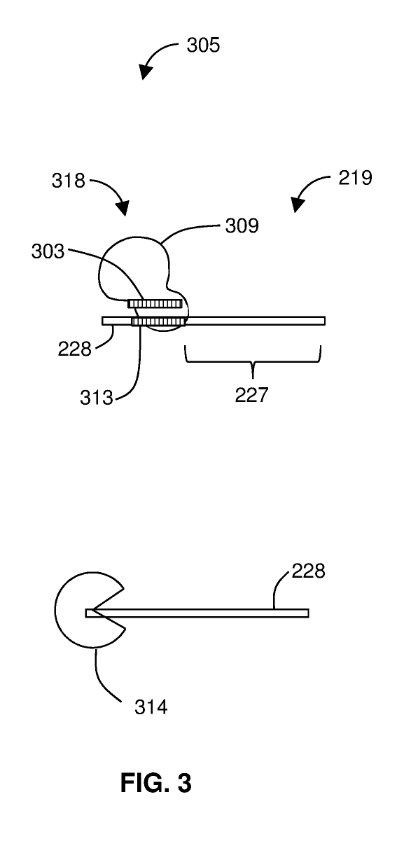

FIG. 3 illustrates a polynucleotide enrichment technique for obtaining a representative portion 227. A biological or clinical sample that contains the target nucleic acid 219 is obtained from a subject or patient. The depicted technique is useful for the isolation of, or enrichment for, a representative portion 227. The technique may use any suitable sample 305. The enrichment technique includes obtaining a blood, plasma, or tissue sample from the patient. Preferably, a sample that includes plasma is obtained. Preferred embodiments include blood, plasma, a tumor sample (such as from a FFPE tumor slice), or any other sample containing nucleic acid. A sequence-specific binder 318 (e.g., a molecule or molecular complex such as Cas9 with gRNA) is introduced to the sample, which binder 318 will bind to a specific target 313. The binding target 313 is adjacent or overlaps the representative portion 227. The representative portion 227 may be of any suitable length and the technique may be used to capture long DNA fragments, including individual fragments with lengths of thousands to tens of thousands of bases. The illustrated technique includes selectively protecting the representative portion 227 while degrading segments 228 of the target nucleic acid 219 other than the representative portion 227.

The binder 318 (e.g., molecules or molecular complexes that interact with the fragment of DNA in a sequence-specific manner) may include, for example, a DNA binding protein, an oligonucleotide, an endonuclease, a transcription-activator like effector (TALE) domain, a TALE nuclease (TALEN), a non-naturally occurring oligonucleotide (e.g., an oligo that includes a conformationally-restricted nucleotide or a phosphorothioate linkage), or any other sequence-specific binder. Thus preferably selectively protecting the at least one fragment includes introducing into the sample at least one molecule that selectively protects the target nucleic acid from digestion in a sequence-specific manner. In a preferred embodiment the molecule 318 or molecular complex includes a Cas endonuclease 309 and a guide RNA 303 that binds to the target 313. A feature of the invention is that active Cas endonuclease is useful to protect the representative portion from nuclease digestion.

The guide RNA 303 and a Cas endonuclease 309 bind to, and protect, the representative portion 227 in the sequence-specific manner. Because the guide RNA 303 complexes with the Cas endonuclease 309 and binds to the target 313 in a sequence-specific fashion, the depicted method may be used to selectively protect the representative portion 227. The segments 228 can then be degraded/ablated. For example, the method may include digesting those segments 228 with an exonuclease 314.

A surprising feature is that catalytically inactive Cas (dCas) may be used, or active Cas may be used. Even when active Cas is used, it will bind to the target 313 and successfully protect the representative portion 227 from the exonuclease 313 (preferably a dsDNA exonuclease). In preferred embodiments, the enrichment technique is used to isolate long a representative portion 227 of, e.g., at least a few kilobases in length.

The described methods may have applicability as a quality control (QC) method for use with an in vivo gene editing procedure. With in vivo gene editing, an enrichment is performed to obtain a representative portion of a target nucleic acid from a subject, such as an organism for whom gene editing will be performed. The representative sample of the subject's nucleic acid may be obtained using a negative enrichment technique in which the enrichment comprises taking a sample from the subject and protecting the representative portion in sequence-specific manner and digesting unprotected nucleic acid. The representative portion may be protected using a gene-editing system. Preferably, the gene editing system comprises programmable nucleases (or nucleic acid encoding the programmable nuclease), and in a most preferred embodiment, each programmable nuclease includes Cas endonuclease (e.g., complexed to a guide RNA specific to part of the representative portion. Exonuclease is introduced into the sample to digest everything but the protected representative portion. The remaining DNA (e.g., the representative portion) is extracted for sequence (e.g., via standard nucleic acid extraction techniques). The in vivo QC method includes sequencing the representative portion to obtain pre-Tx sequence reads (aka expected sequence). The gene editing procedure is performed on the subject. After the gene editing is performed on the subject, another enrichment is performed to obtain a post-treatment representative portion.

The in vivo QC technique includes sequencing the post-treatment representative portion to obtain post-Tx sequence reads. The post-Tx sequence reads are compared to the pre-Tx sequence reads to determine an off-target rate. In an exemplary embodiment, any significant difference between the pre-Tx sequence reads and the post-Tx sequence reads counts as evidence of an instance of off-target activity. The evinced instances of off-target activity may be counted to determine an off-target rate for the representative portion. The in vivo QC method may preferably include inferring an off-target rate for the target nucleic acid based on the determined off-target rate for the representative portion.

By reporting the off-target rate for gene editing that was performed in vivo, one may use the described methods to validate a success or potential issues with gene editing.

In some embodiments, the representative sample comprises one or more pre-determined fragments of test nucleic acid provided in a kit.

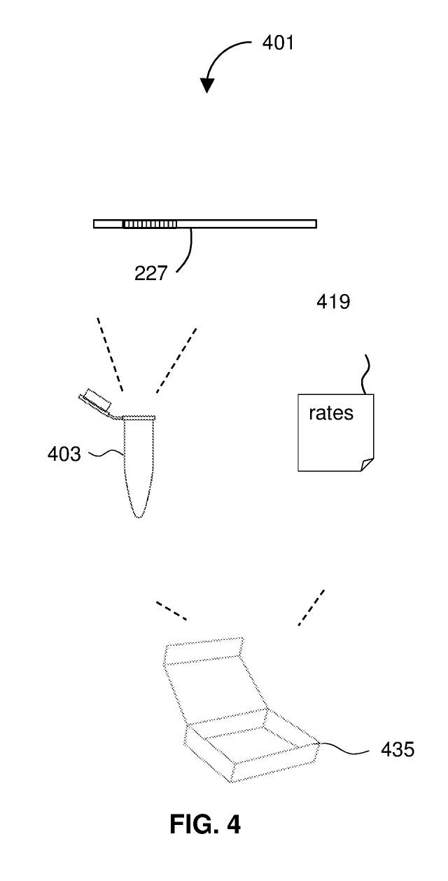

FIG. 4 illustrates a kit 401 for quality control according to one embodiment. The kit 401 may include a representative portion 227 of the target nucleic acid 219 or reagents for obtaining the representative portion 227. The representative portion 227 or reagents may be provided in a suitable container, optionally with instructional information 419, which may be a chart or instructions for conversion of rates of off-target activity with representative sample 227 to off-target activity for a whole biological sample. Thus, the kit 401 may include instructions for determining an off-target rate of a gene editing process on the representative sample and reporting an off-target rate of the gene editing process on the subject nucleic acid. Any components of the kit 401 may be packaged in container to facilitate shipping to clinics and labs for the performance of standardized quality control operations.

Embodiments of the invention include using the kit 401 to report an off-target rate of binding of the Cas endonuclease to the subject nucleic acid based on the determined off-target rate for the process with the representative sample represents. Determining the off-target rate includes performing an assay to show an amount of off-target activity resulting from introduction of the material to the representative sample.

Methods and kits of the invention are useful not just to determine an off-target rate of activity, but to identify a set of reaction conditions at which the off-target rate meets a desired threshold (such as zero). To illustrate, if it sought to perform gene-editing in an organism to remove a gene, it may be beneficial to show that with certain reagents and reaction conditions, removal of the gene is performed without off-target activity. A gene editing system, such as a Cas endonuclease and appropriate guide RNA(s) may be provided, as well as solutions, equipment, and relevant reagents. A sample from the subject organism containing representative nucleic acid may be obtained, and—in a first trial—Cas with guide RNA may be delivered to the representative sample. Using methods described herein (e.g., a heteroduplex assay or PAGE), an off-target rate is determined. It may be found that a non-zero and unacceptable off-target rate obtains. Conditions may be varied, and another a trial may be performed. For subsequent trials, any suitable conditions may be varied. For example, various trials could be performed at different concentrations of Cas endonuclease (or nucleic acid encoding the same), guide RNA(s), salt, metal ions, etc. Temperature may be varied. The guide RNAs or the nuclease may be changed. For example, different Cas homologs may be tested. Different delivery vectors or methods may be tried. For example, Cas delivered by solid lipid nanoparticles may be compared to Cas delivered by liposome, which may be compared to the delivery of naked Cas. Performing multiple trials allows one to identify not only those conditions under which an acceptable rate of off-target activity is exhibited, but also to optimize for other variables such as reagent cost or required packaging complexity, or user difficulty. Thus, embodiments of the method 101 include conducting at least a first trial, second trial, and third trial of introducing the material to the representative sample, each trial comprising unique conditions. Each trial is used for showing a respective off-target rate for that trial. A set of conditions associated with an off-target rate shown to be below a threshold is thus identified and accepted as the acceptable conditions for the prospective gene-editing assay going forward.

Reference is made to a level of off-target activity meeting a desired threshold. Any suitable threshold may be met using methods of the invention. For example, in some embodiments, a threshold is set by a third party. For example, a research firm contracting to develop genetically modified agricultural seeds for an agricultural products corporation may have the threshold set by the agricultural products corporation (e.g., the company can bring the seed to market if made using Cas9 with no greater than 5% off-target rate generally across a soybean genome). The threshold may be set by a government regulatory agency. The threshold may be zero. Preferably, the threshold is a predetermined threshold set, e.g., by a third-party, to ensure that the process performed under the selected conditions will not exhibit a significant off-target rate when performed on the subject nucleic acid.

Methods of the invention may optionally include demonstrating that the sample is representative of the plurality of genetic loci. For a DNA sample to be representative of a nucleic acid of interest, it may be understood to mean that a count of off-target activity in the representative sample can be reliably correlated to off-target activity in a whole genome. The sample may be demonstrated to be representative by any suitable method. Suitable methods may include whole genome sequencing, genome-wide association studies, linkage disequilibrium analyses, or Bayesian regression.

Some embodiments include linkage disequilibrium analysis. Linkage disequilibrium is the non-random association of alleles at different loci in a given population. Loci are said to be in linkage disequilibrium when the frequency of association of their different alleles is higher or lower than what would be expected if the loci were independent and associated randomly. Linkage disequilibrium may exist between alleles at different loci without any genetic linkage between them and independently of whether or not allele frequencies are in equilibrium (not changing with time). It may suffice for being a representative sample that a statistical distribution of loci in linkage dis-equilibrium is not significantly different than the statistical distribution of linkage disequilibrium over the human genome. Additionally or alternatively, it may suffice for a representative sample that the representative sample simply does not include any substantial hotspots of linkage disequilibrium. Without being bound by any mechanism, it may be theorized that arbitrary small fractions of a human genome (such as a single exon, or an established panel of genes associated with heredity disorders) are not themselves useful as representative samples because they are inherently hotspots for linkage disequilibrium.

For example, it is understood that many recessive hereditary disorders are resultant of founder effects and associated population bottlenecks. Such disorders are of great clinical significance and it may be beneficial to address such conditions with gene therapy or gene editing tools. Unfortunately, phenomena such as founder effects and population bottlenecks defy the predicates of Harvey-Weinberg equilibrium, and thus people affected with those conditions may exhibit the greatest linkage disequilibrium at the affected loci. For those reasons, isolated whole genes or panels of whole genes for those conditions may be very poor representative samples of the genome at large. Thus for gene editing for hereditary disorders in particular, it may be the most beneficial to ensure a representative sample by, for example, establishing that the prospective sample does not exhibit a degree of linkage disequilibrium that otherwise characterizes the loci associated with the hereditary disorder. The same may be true for cancer, as tumor driver mutations (potentially arising from the most extreme bottleneck of a population size equal to one) may be extreme hotspots for linkage disequilibrium. In such cases, without quality control methods of the disclosure, gene editing materials addressed to oncogenes, tumor DNA, or other cancer markers may otherwise exhibit off-target activity far what would be expected by probability.

Certain embodiments include Bayesian methods for determining representativeness of a selection of loci. A Bayesian hierarchical model may be implemented to learn an informative prior distribution from sequence features. See Huang, 2017, BRIE: transcriptome-wide splicing quantification in single cells, Genome Biology 18(1):123, incorporated by reference. Bayesian Regression for Isoform Estimation (BRIE) is a statistical model that achieves extremely high sensitivity at low coverage by using informative priors learned directly from data via a (latent) regression model. The regression model couples the task of splicing quantification across different genes, allowing a statistical transfer of information from well-covered genes to less well covered genes, achieving considerable robustness to noise in low coverage. BRIE can be implemented to show that a portion of a target nucleic acid is representative of the target nucleic acid. BRIE can provide a reliable and reproducible method to quantify off-target active across genomes.

Embodiments of the invention may be used for quality control of sequence-specific treatments of nucleic acids generally. For any entity or material that binds to, or interacts with, a nucleic in a sequence-specific fashion, methods and kits may be used to determine an off-target rate of binding of the material with a representative sample, to predict an off-target rate for a subject nucleic acid such as a whole genome.

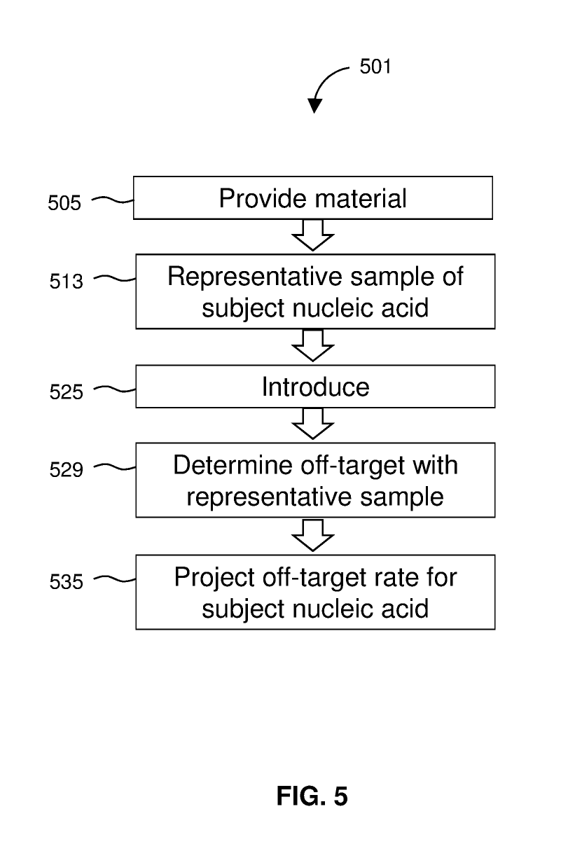

FIG. 5 diagrams a quality control method 501. The method includes providing 505 material designed to interact with a target in a subject nucleic acid. A representative sample for the subject nucleic acid is obtained 513. The method 501 includes introducing 525 the material to the representative sample, and determining 529 an off-target rate of interaction of the material with the representative sample. The method 501 may be used for projecting 535 an off-target rate of interaction between the material and the subject nucleic acid based on the determined off-target rate of interaction of the material with the representative sample. The material can be any suitable sequence-specific binding element and is preferably a molecule or molecular complex. Exemplarily molecular entities for the material include DNA-binding proteins, oligonucleotides (include DNA, RNA, modified DNA or RNA, peptide nucleic acids, etc), restriction enzymes, probes, or other suitable materials.

To illustrate an exemplary embodiment of method 501, if a researcher intended to use gene editing to introduce or correct mutations in a gene such as for a hereditary disorder such as cystic fibrosis transmembrane conductance regulator (CFTR), the researcher could select a vial from the kit in which the representative sample is entirely derived from human genome, chromosome 1 (CFTR being located on Ch3). The assay could be performed to demonstrate that the control ladder and the trial ladder were exactly matched, thus showing that the gene editing materials exhibited no off-target effects with the representative sample. Thus, the methods are useful for quality control for such prospective gene-editing reagents.

A representative sample may be any suitable subset of, or surrogate for, a subject nucleic acid of interest. In some embodiments, methods of the invention include enriching for a representative sample from a larger sample, e.g., the subject nucleic acid. Similarly, kits of the invention may include reagents useful for isolating or enriching for a representative sample. Such methods and kits may employ sequence-specific protections of target nucleic acid (e.g., to be the representative sample) and ablation of non-target. Such kits and methods may include gene editing materials. In some embodiments, the method 101 uses a material designed to interact with a target (e.g., gene editing materials) in a subject nucleic acid as well as a representative sample for the subject nucleic acid. Methods and kits of the invention may be used to quality control the gene editing materials.

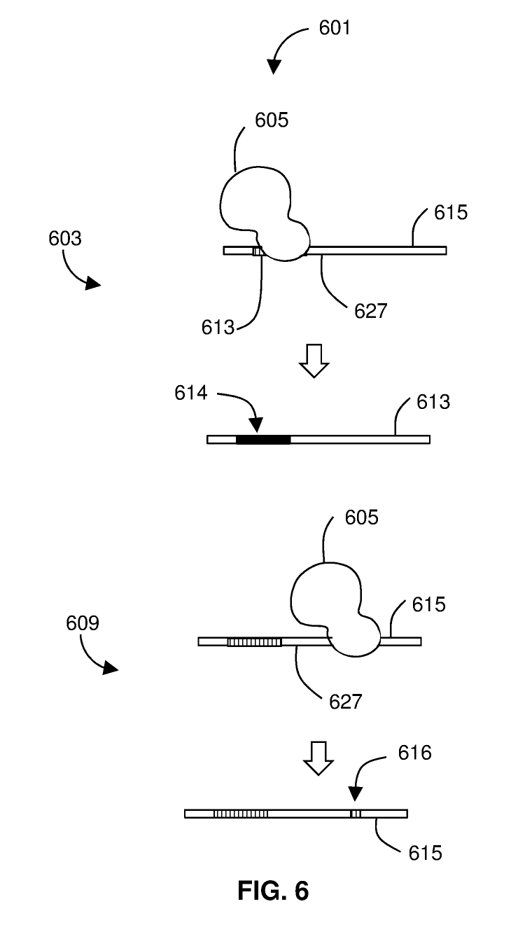

FIG. 6 illustrates an assay 601 of on-target interaction 603 of a material 605 with the representative sample 627 as well as off-target interaction 609 of the material 605 with the representative sample 627. On-target interaction 603 yields an expected product 613. The expected product 613 includes the intended change 614.

In contrast, off-target interaction 609 yields an off-target product 615. In the depicted embodiment, off-target product 615 includes an undesired change 616. It is of interest to quantify off-target activity 609, where the Cas endonuclease binds distal from the target 613 and any activity yields off-target product. Thus when the material 605 is being assayed with the representative sample 627, the resultant products 613, 615 can be assayed to determine a rate of off-target activity. The resultant products 613, 615 can themselves be directly assayed, or those products may be fragmented, or portions of those products may be amplified, and resultant fragments or amplicons may be assayed for off-target activity (e.g., by detecting an amount of undesired change 616).

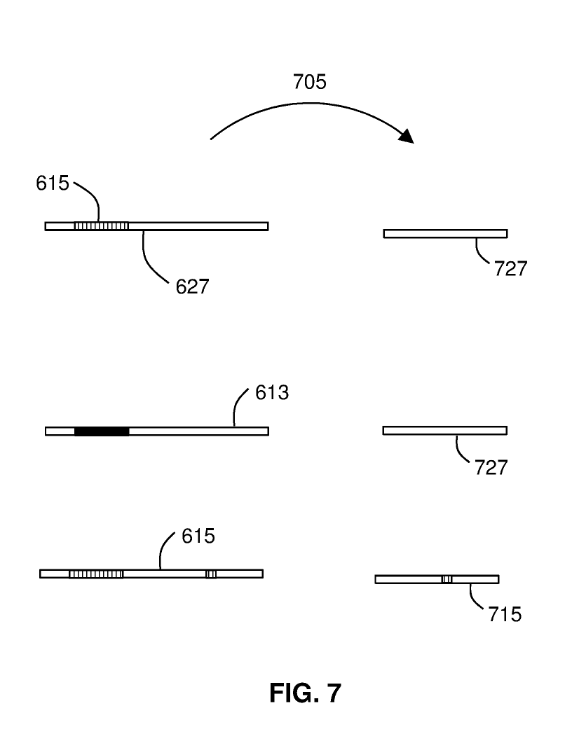

FIG. 7 shows the result of fragmenting or amplifying 705 products of the assay 601. In preferred embodiments, the fragmenting or amplifying 705 captures a segment of the representative sample 627 that does not include the target 613. The assay 601 may yield one or more of an expected product 613, an off-target product 615, and the representative sample 627. Any portions of the representative sample 627 that were unaffected by the assay 601 will yield WT fragments 727 after any fragmentation or amplification 705. Any portion of the representative sample 627 that was correctly edited will include expected product 613, and fragmentation or amplification 705 of the expected product 613 will also yield WT fragments 727. Any portion of the representative sample 627 that experienced off-target activity will yield off-target product 615, and fragmentation or amplification 705 of the off-target product 615 will yield mutant fragments 715. To summarize from assay 601 and amplification 705:

Sample 627→no activity→sample 627→amplify 705→WT fragments 727;

Sample 627→on target 603→product 613→amplify→WT fragments 727; and

Sample 627→off-target 609→off-target prod 615→amplify 705→mutant frags 715.

Determining an off-target interaction may include comparing the portions of the off-target product 615 to portions from the expected product 613 and the representative sample 627. Specifically, WT fragments 727 may be obtained from the expected product 613, the representative sample 627, or both. Similarly, mutant fragments 715 may be obtained from the off-target product 615.

It is noted that determining the off-target activity can be performed on the raw targets from the cleavage assay with gene-editing materials, or the determination can be performed with fragments of those products. It may be preferable in embodiments to test for off-target activity by inspection of only fragments of the raw product. Those fragments may be obtained in any suitable way such as by a restriction endonuclease digest, or any obtained mixture the off-target product 615, the expected product 613, and the representative sample 627 may be tested directly. Use of fragments may be most preferable where an intended target is flanked by primer binding sites (duplicates of which may be elsewhere in the representative sample). For example, the representative sample may be provided by a kit that includes aliquots of substrate fragments with primer binding sites, some of which include a prospective gene editing target and some of which do not. After incubation with the gene editing materials, primers and polymerase may be used to amplify fragments from those aliquots. Such an assay may yield a mixture of WT fragments 727 and mutant fragments 715.

Those fragments may then readily be assayed to determine off-target activity.

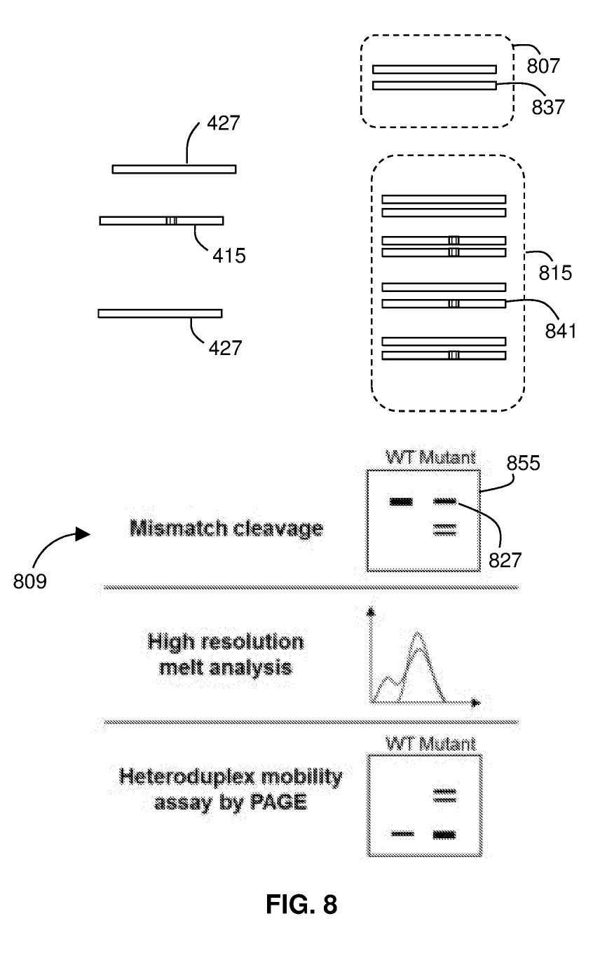

FIG. 8 illustrates determining off-target rate from the products present after on-target interaction 603 of the material 605 with the representative sample 627 as well as off-target interaction 609 of the material 605 with the representative sample 627. Where only on-target interaction 603 of the material 605 with the representative sample 627 occurs, a first product pool 807 results. Where off-target interaction 609 of the material 605 with the representative sample 627 occurs, a second product pool 815 results. Determining 129 the off-target rate of interaction of the material with the representative sample may be done by measuring a relative quantity of homoduplexes 837 and heteroduplex 841 in the product pool. For example, 100 percent homoduplexes is evidence of no off-target activity.

Any suitable method may be used for determining the off-target rate with the representative sample. Any suitable test may be performed to detect off-target activity from the mixture of WT fragments 727 and mutant fragments 715. Suitable assays may include PCR, qPCR, rtPCR, digital PCR, probe hybridization assays (such as SNP chips) or probe capture, allele-specific ligation or amplification assays, mismatch cleavage In some embodiments, the off-target rate is determined by sequencing a product of the process; a heteroduplex cleavage assay; a high-resolution melt curve; qPCR; and a heteroduplex mobility assay. Preferred approaches include a high-resolution melt analysis, a heteroduplex mobility assay (e.g., by PAGE), or an assay for mismatch cleave. Other methods for determining the off-target rate may include high-resolution melt analysis (HRMA), cleaved amplified polymorphic sequencing (CAPS), loss of primer binding site, amplified fragment length polymorphism (AFLP), and fluorescent PCR-capillary gel electrophoresis. For background, the determining 129 may include methods described in Zischewski, 2017, Detection of on-target and off-target mutations generated by CRISPR/Cas9 and other sequence-specific nucleases, Biotechnology Advances 65:95-104, incorporated by reference. In certain embodiments, determining 129 the off-target rate includes mismatch cleavage assay 809.

A mismatch cleavage assay 809 is useful for the detection of indels induced by genome editing. The assay uses enzymes that cleave heteroduplex DNA at mismatches and indel-associated loops. Distinct PCR products are generated by the cleavage and the resulting fragments may be detected by gel electrophoresis or high-performance liquid chromatography (HPLC). Such assays may be automated and may take an hour or less. Any suitable enzyme may be used. One such enzyme is T7 endonuclease 1 (T7E1), which cleaves mismatched DNA at the first, second or third phosphodiester bond upstream of the mismatch. Another such enzyme is the CEL-family endonuclease sold under the trademark SURVEYOR by Transgenomic, Inc. (Omaha, Nebr.). The CEL-family endonuclease cleaves in the presence of SNPs or small indels, cleaving both DNA strands downstream of the mismatch. T7E1 provides sensitivity for indels generally, while the CEL-family endonuclease is good for SNPs and very small indels. It may be preferable to add WT DNA to encourage the formation of hetero-duplexes to aid in the determination 129 of the off-target rate. In performing the mismatch cleavage assay 809, PCR products 827 that are generated by the cleavage and the resulting fragments may be detected by gel electrophoresis on a polyacrylamide gel 855, or by some such similar assay. In certain embodiments, the determined off-target rate of interaction of the material with the representative sample is used for projecting an off-target rate of interaction between the material and the subject nucleic acid.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

### EXAMPLES

### Example 1

### Cas9 QC

For each independent CRISPR/Cas9 experiment, existing approaches require that WGS be performed on the targeted genome of interest before and after gene editing, to determine an extent of off-target events. While such an approach theoretically generates an absolute off-target rate for each Cas9/gRNA complex used in an experiment, it is observed that, within the sequencing field, there are a number of variables (e.g., sample prep, library production, depth of coverage, bioinformatic analysis) that can significantly differ among labs and researchers. To address such variance, standardized methods, specifications, kits, and samples are provided for quality control and the control analysis.

Embodiments of the solution include the development of universal low cost CRISPR associated QC kits and methods. Such a kit may include a representative sample of a subject nucleic acid or reagents for obtaining the representative sample as well as instructions for determining an off-target rate of a gene editing process on the representative sample and reporting an off-target rate of the gene editing process on the subject nucleic acid.

Such a method may include introducing a Cas endonuclease and at least one guide RNA to the representative sample; determining the off-target rate for binding of the Cas endonuclease and the guide RNA to the representative sample; and reporting an off-target rate for binding of the Cas endonuclease and the guide RNA to the subject nucleic acid based on the determined off-target rate for binding of the Cas endonuclease and the guide RNA to the representative sample.

### Example 2

### Enrichment for a Representative Sample

In some embodiments, kits and methods of the invention are used as a quality control in a process of enriching a target nucleic acid from a sample. An enrichment may use some material such as a sequence-specific binding molecule or complex designed to interact with the target to perform enrichment of a target nucleic acid from the subject nucleic acid.

The enrichment may include protecting the target nucleic acid in sequence-specific manner and cleaving unprotected nucleic acid, and further wherein determining the off-target rate for the process shows that only the target nucleic acid survives the cleaving step.

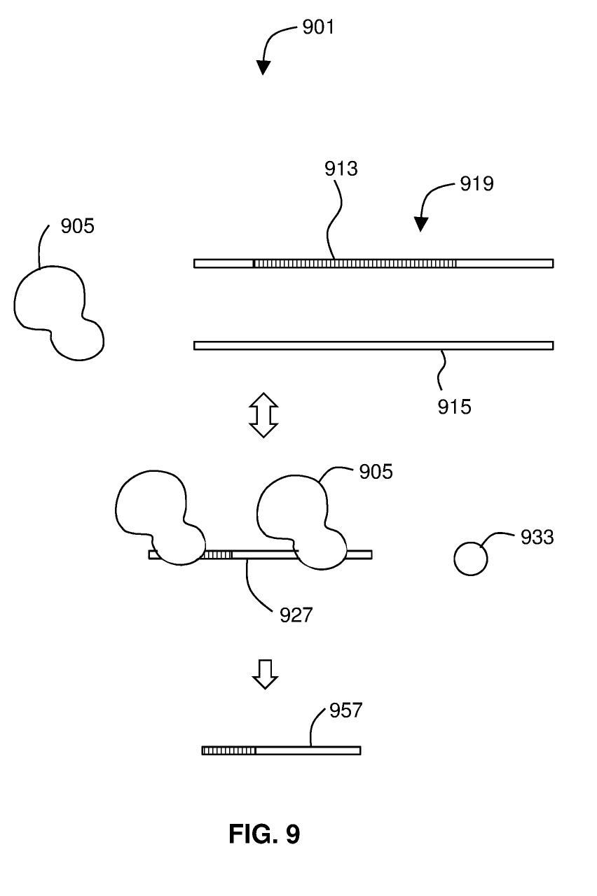

FIG. 9 shows a method 901 for polynucleotide enrichment using a programmable nuclease, usable as quality control step for a proposed a CRISPR/Cas-mediated polynucleotide enrichment. Specifically, the method 901 demonstrates an off-target rate for the proposed use of the programmable nuclease 905, such as a Cas endonuclease. In embodiments of the method 901, a nucleic acid region of interest 913 is within or among subject nucleic acid 919. The subject nucleic acid 919 includes the nucleic acid region of interest 913 as well as any number of off-target regions 915. The programmable nuclease 905 binds specifically at sequences 3′ and 5′ of the nucleic acid region of interest 913. Thus, the subject nucleic acid 919 is selectively blocked from nuclease digestion by the binding of the nuclease 905 (e.g., CRISPR/Cas complexes) at sequences 5′ and 3′ to the region of interest.

Where the nuclease 905 is a Cas endonuclease, specific guide RNAs direct CRISPR/Cas proteins to sequences that flank a nucleic acid molecule region of interest 913 on both its 5′ and 3′ ends. Binding of the CRISPR/Cas complexes to the nucleic acid molecule provides a mechanical shield from nuclease-mediated digestion, such exonuclease-mediated digestion. Subsequent exposure of the nucleic acid mixture to a nuclease, such as an exonuclease, results in digestion of the unprotected nucleic acid molecules and, thus, enrichment of the region of interest.

In some embodiments, enrichment of a nucleic acid region of interest comprises protecting the region of interest by contacting the nucleic acid molecule with at least two different guide RNAs and CRISPR/Cas proteins, optionally catalytically-inactive CRISPR/Cas proteins, wherein each of the two different guide RNAs binds to a different sequence of the nucleic acid molecule flanking the region of interest and wherein the CRISPR/Cas proteins bind to the nucleic acid molecule at the guide RNAs, and contacting the nucleic acid molecule with the bound guide RNAs and CRISPR/Cas proteins with an exonuclease to digest the nucleic acid molecule 5′ and 3′ to the bound guide RNAs and CRISPR/Cas proteins, thereby digesting the nucleic acid molecule outside of the region of interest.

As used herein, the term “CRISPR/Cas protein” or Cas endonuclease includes RNA-guided DNA endonucleases, including, but not limited to, Cas9, Cpf1, CasX, CasY C2c1, and C2c3 and each of their orthologs and functional variants.

In some embodiments, a representative sample 927 for the subject nucleic acid 919 is used to predict an off-target rate of binding of the nuclease 905 when used in the enrichment method 901. As well as performing the enrichment steps described above, one or more copies of the nuclease 905 (or nucleic acid encoding the same) are introduced to the representative sample 927. The nuclease 905 is incubated with the representative sample, allowing for an off-target binding to occur. An exonuclease 933 is introduced, resulting in promiscuous digestion of unbound portions of the representative sample. It may be found that it is moot whether or not the nuclease 905 cleaves the subject nucleic acid 919 at the target site of the nucleic acid region of interest 913. Without being bound by any theory, it may be that the nuclease 905 stays bound to the nucleic acid 919 (with or without cleavage thereof) for a duration sufficient to allow the exonuclease to exhibit its full range of canonical activity, digestion of unprotected DNA. After digestion, the sample is assayed for the presence of DNA fragments 857 incompatible with fully on-target binding by the nuclease 905.

Any suitable method may be used to assay for the presence, or quantity, of fragment(s) 957. Suitable methods may include PCR, qPCR, spectrophotometry, fluorescent probes, or other methods known in the art. The detected presence or quantity of the fragments 957 correlates to a rate of off-target binding by the nuclease 905.

The enrichment method 901 may be used to enrich a long fragment of a target nucleic acid from a mixed sample or biological sample. The method may be used to attach to, and isolate, long fragments including fragments of thousands, tens of thousands, hundreds of thousands of bases in length or longer. The target is enriched by virtue of the fact that the exonuclease digests away any number of off-target regions 915. Since the resultant sample only includes long fragments of target nucleic acid, that sample may be particularly suited for certain sequencing or other analytical techniques (and particularly for long-read, single molecule sequencing). Since the off-target rate of the nuclease 805 is determined in the process, confidence may be had that the resultant sample only contains the target. This allows a number of benefits including, for example, the promiscuous ligation of sequencing adapters and/or universal priming sites, which may facilitate the downstream analysis.

Kits and methods of the invention are useful with methods disclosed in U.S. Provisional Patent Application 62/526,091, filed Jun. 28, 2017, for NUCLEIC ACID MOLECULE ENRICHMENT MOLECULES and U.S. Provisional Patent Application 62/519,051, filed Jun. 13, 2017, for NUCLEIC ACID MOLECULE ENRICHMENT METHODOLOGIES, both incorporated by reference.

One property of those methods is that little sample manipulation is needed to prepare the DNA for sequencing and analysis. The minimal sample preparation should have an immediate impact on the intra- and inter- lab variability associated with the number of sample manipulations needed to generate results. This is especially important due to the fact that the “control” aspects of each Cas9 experiment involve a pre- and post Cas9 treatment.

What is claimed is:

1. A method comprising:
treating a representative portion of a target nucleic acid with a gene editing system to yield a nucleic acid product;
sequencing the nucleic acid product to produce sequence reads;
determining an off-target rate for the representative portion by comparing the sequence reads to predicted sequence that would result from treatment by the gene editing system with no off-target activity; and
inferring an off-target rate for the target nucleic acid based on the determined off-target rate for the representative portion.

2. The method of claim 1, wherein the target nucleic acid includes a target of the gene editing system, and wherein the representative portion does not include the target.

3. The method of claim 1, further comprising sequencing the representative portion prior to the treating step to produce the sequence corresponding to no off-target activity.

4. The method of claim 1, further comprising performing, prior to the treating step, an enrichment to obtain the representative portion from the target nucleic acid.

5. The method of claim 4, wherein the enrichment comprises protecting the representative portion in sequence-specific manner and digesting unprotected nucleic acid.

6. The method of claim 1, wherein the representative portion comprises one or more pre-determined segments of target nucleic acid provided in a kit.

7. The method of claim 1, wherein the gene editing system comprises at least one programmable nuclease or a nucleic acid encoding the programmable nuclease.

8. The method of claim 7, wherein the programmable nuclease comprises a Cas endonuclease.

9. The method of claim 1, further comprising:
conducting at least a first trial, second trial, and third trial of treating the representative portion with the gene editing system, each trial comprising unique conditions;
showing a respective inferred off-target rate for each trial; and
selecting a set of conditions associated with an off-target rate shown to be below a threshold.

10. The method of claim 9, wherein the threshold is a predetermined threshold indicating that the gene editing system used under the selected conditions will not exhibit a significant off-target rate when performed on the target nucleic acid.

11. The method of claim 1, further comprising obtaining the representative portion from the target nucleic acid by ablation of portions of the target nucleic acid that are not comprised by the representative portion.

12. The method of claim 1, wherein the representative portion is obtained from the target nucleic acid by a process that is agnostic of genes or coding regions within the target nucleic acid.

13. The method of claim 1, wherein the representative portion is less than the target nucleic acid.

14. The method of claim 1, further wherein the method is used as a quality control for a use of Cas9, and the method comprising:
introducing a Cas9 endonuclease and at least one guide RNA to the representative portion;
determining the off-target rate for binding of the Cas9 endonuclease and the guide RNA to the representative portion; and
reporting an off-target rate for binding of the Cas9 endonuclease and the guide RNA to the target nucleic acid based on the determined off-target rate for binding of the Cas9 endonuclease and the guide RNA to the representative sample.

15. A method comprising:
treating a representative portion of a target nucleic acid with a gene editing system to yield a nucleic acid product;
determining an off-target rate for the representative portion by performing an assay on the nucleic acid product; and
inferring an off-target rate for the target nucleic acid based on the determined off-target rate for the representative portion.

16. The method of claim 15, wherein the representative portion is less than the target nucleic acid.

17. The method of claim 16, wherein the gene editing system comprises a Cas9 endonuclease and a guide RNA.

18. The method of claim 17, further comprising obtaining the representative portion from the target nucleic acid by ablation of portions of the target nucleic acid that are not comprised by the representative portion.

19. The method of claim 18, wherein the method is used as a quality control process for Cas endonuclease to show an acceptable rate of off-target activity by the Cas endonuclease with the target nucleic acid.

20. The method of claim 19, wherein the target nucleic acid includes a human genome.

21. The method of claim 20, wherein determining the off-target rate includes one selected from the group consisting of: sequencing a product of the process; a heteroduplex cleavage assay; a high-resolution melt curve; qPCR; and a heteroduplex mobility assay.

22. A kit for quality control, the kit comprising:
a representative sample of a subject nucleic acid or reagents for obtaining the representative sample; and
instructions for determining an off-target rate of a gene editing process on the representative sample and reporting an inferred off-target rate of the gene editing process on the subject nucleic acid.
